(19) United States
(12) Patent Application Publication
(10) Pub. No.: US 2005/0288694 A1
(43) Pub. Date: Dec. 29, 2005

(54) Adjustable percutaneous stomach lumen restriction device

(76) Inventor: Stepehen Solomon, New York, NY (US)

Correspondence Address: FRISHAUF, HOLTZ, GOODMAN & CHICK, PC, 220 5TH AVE FL 16, NEW YORK, NY, 10001-7708, US

(21) Appl. No.: 11/165,632

(22) Filed: Jun. 23, 2005

Related U.S. Application Data
(60) Provisional application No. 60/582,143, filed on Jun. 23, 2004.

(51) Int. Cl. A61B 17/08

(52) U.S. Cl. 606/157

(57) ABSTRACT

A device is provided for percutaneously restricting a stomach lumen in which an outer casing surrounds a needle to define a space between the outer casing and the needle. A suture is provided in the space between the outer casing and the needle, and a plurality of disks are provided along the suture. The needle is adapted to pierce a wall of the lumen at a plurality of positions. The outer casing is retractable with respect to the needle to provide an opening through which the disks individually exit the space between the outer casing and the needle at each of the plurality of positions. And the lumen is restricted by drawing together the disks along the suture.

### CROSS REFERENCE TO RELATED APPLICATION

The present application is based upon and claims the benefit of priority of provisional application Ser. No. 60/582,143, filed on Jun. 23, 2004.

### FIELD OF THE INVENTION

The present invention relates to methods and apparatuses for percutaneously restricting the gastric lumen.

### BACKGROUND OF THE INVENTION

Obesity affects many individuals around the world, and morbid obesity (characterized by a Body Mass Index (BMI) of at least 40 kg/m² or by a BMI of at least 35 kg/m² with the presence of one or more comorbidities) in particular is associated with serious health problems, both physical and psychological, including diabetes, high blood pressure, gastroesophageal reflux disease, sleep disorders, asthma, depression and sexual dysfunction. While nonsurgical weight loss strategies, such as reducing caloric intake or modifying behavior, can be effective in reducing weight in the short term, nonsurgical weight loss methods are associated with only minimal, and temporary, weight loss. Therefore, long-term management of morbid obesity is typically obtained through surgery.

Surgical weight loss methods can be categorized as either (or both) of restrictive, in which the amount of food that can be eaten is reduced by reducing the capacity of the stomach, or malabsorptive, in which the ability of the intestine to digest food is reduced. Two common surgical procedures are the Roux-en-Y gastric bypass and vertical banded gastroplasty. In the Roux-en-Y gastric bypass procedure, the stomach is divided to form a small proximal pouch, which is attached directly to, for example, the jejunum, thereby bypassing most of the intestine. In vertical banded gastroplasty, the stomach is stapled to create a small pouch; no rerouting of the intestine is performed. In both procedures, the reduced stomach capacity causes the patient to feel “full” after eating less food.

Although most weight loss surgeries can be performed laparoscopically, they tend to be complex. In addition, procedures such as the Roux-en-Y gastric bypass and vertical banded gastroplasty are invasive and not easily reversible.

Laparoscopic adjustable gastric banding presents a less invasive, adjustable and more easily reversible alternative to both Roux-en-Y gastric bypass and vertical banded gastroplasty. In laparoscopic adjustable gastric banding an inflatable band is inserted laparoscopically and buckled around the stomach to create a small proximal pouch. An access port is positioned subcutaneously such that saline may be injected (or removed) by a needle into the access portion to inflate (or deflate) the band around the stomach. The size of the stoma between the proximal and distal portions of the stomach may thereby be adjusted via the injection and removal of saline through the access port. Laparoscopic adjustable gastric banding has been shown to be safer than both Roux-en-Y gastric bypass and vertical banded gastroplasty, at least in the short term. (See Chapman et al, “Laparoscopic adjustable gastric banding in the treatment of obesity: A systematic literature review,” Surgery, Vol. 135 No. 3, March 2004, 326-351).

Stomach restriction may also be performed by apposing sides of the stomach together endoscopically, as described in U.S. Pat. No. 6,558,400.

It should be possible to further reduce the risks and costs associated with gastric banding by narrowing the stomach lumen percutaneously.

### OBJECT OF THE INVENTION

It is an object of the present invention to provide a method and apparatus for percutaneously narrowing the stomach lumen.

### SUMMARY OF THE INVENTION

According to one aspect of the present invention a device is provided for percutaneously restricting a lumen, which includes a needle, an outer casing which surrounds the needle to define a space between the outer casing and the needle, a suture provided in the space between the outer casing and the needle, and a plurality of disks provided along the suture. The needle is adapted to pierce a wall of the lumen at a plurality of positions. The outer casing is retractable with respect to the needle to provide an opening through which the disks individually exit the space between the outer casing and the needle at each of the plurality of positions. And the lumen is restricted by drawing together the disks along the suture.

According to another aspect of the present invention, a method is provided for percutaneously restricting a lumen, which includes piercing a wall of the lumen from within the lumen at a plurality of positions; depositing a disk outside of the wall of the lumen at each of the plurality of positions, the disks being connected along a suture; and restricting the lumen by drawing together the disks along the suture.

According to another aspect of the present invention, a device is provided for percutaneously restricting a stomach lumen, which includes a first catheter comprising a first inflatable balloon portion at a distal end thereof, and a second catheter comprising a second inflatable balloon portion at a distal end thereof. The second catheter is adapted to be coupled to the first catheter such that when the second catheter is coupled to the first catheter the first balloon portion and the second balloon portion are provided at an adjustable distance with respect to each other along a lengthwise direction of the catheters.

### BRIEF DESCRIPTION OF THE DRAWINGS

FIG. 1 shows a device for percutaneously restricting the stomach lumen according to a first embodiment of the present invention;

FIG. 2 shows a disk for use with the device according to the first embodiment;

FIG. 3 shows a modification of the device according to the first embodiment;

FIG. 4 shows a groove for use in a groove-and-projection connection in the device according to the first embodiment;

FIG. 5 shows a modification of the device according to the first embodiment;

FIG. 6 shows a modification of the outer casing of the device according to the first embodiment;

FIG. 7 shows the device according to the first embodiment piercing through the near and far walls of the stomach of a patient;

FIG. 8 shows the device according to the first embodiment depositing a disk outside the stomach of the patient;

FIGS. 9A and 9B show a final disk sliding down the suture to the stomach and a stomach lumen restricted using the device according to the first embodiment;

FIGS. 10-13 show the use of a trocar to position a wire through both the near and far walls of the stomach of a patient, for us in positioning a device for percutaneously restricting the stomach lumen according to a second embodiment of the present invention;

FIG. 14 shows the positioning of a first catheter of the device according to the second embodiment;

FIG. 15 shows the first catheter in detail;

FIG. 16 is an enlarged view of the distal end of the first catheter;

FIG. 17 is an enlarged view of the distal end of the first catheter when the balloon at the distal end thereof is inflated;

FIG. 18 shows the first catheter in position in the patient;

FIGS. 19-21 show a second catheter of the device according to the second embodiment being screwed onto the first catheter; and

FIG. 22 shows the device according to the second embodiment in place in a patient.

### DETAILED DESCRIPTION

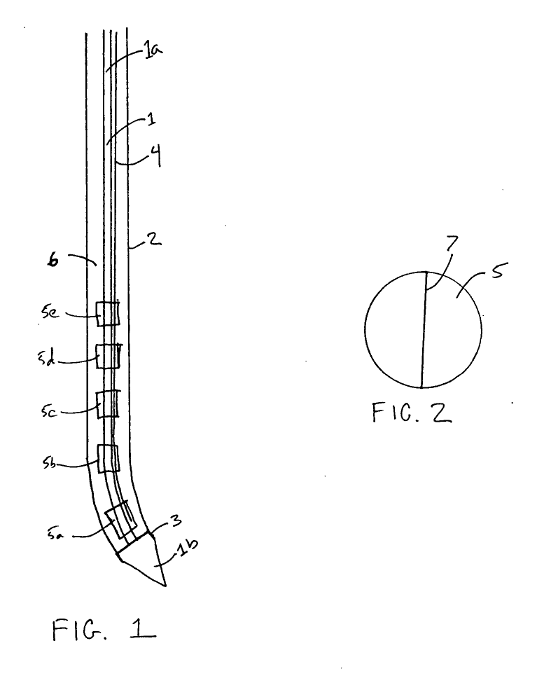

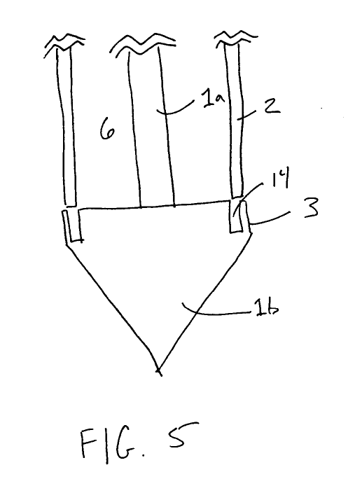

As shown in FIG. 1, a device for percutaneously restricting the stomach lumen according to a first embodiment of the present invention will include disks 5 (5a-5e), which, as described in detail hereinbelow, will be deposited on the outside of the stomach with the suture 4 extending therebetween to restrict the lumen of the stomach (see FIG. 4).

FIG. 1 shows needle 1 surrounded by tubular outer casing 2. The distal end of the outer casing 2 will be attachable to the needle 1 at a distal connection portion 3 of the needle 1. While not connected onto the needle 1, the outer casing 2 will be retractable from the distal end of the needle 1. As shown in FIG. 1, the tip section 1b of the needle 1 will be broader than the shaft 1a. And as shown in FIG. 1, the distal connection portion 3 will be provided at a proximal part of the tip section 1b. In this manner a space 6 will be defined between the needle shaft 1a and the outer casing 2, behind the tip section 1b. Thus, when the outer casing 2 is attached to the needle 1 at the distal connection portion 3, the space 6 will be sealed, and when the outer casing 2 is detached and retracted from the connection portion, a part of the space 6 will be exposed.

Suture 4 will be provided in the space between the needle 1 and the outer casing 2, and will have disks 5 (5a-5e) attached thereto at intervals of a few centimeters such that the suture 4 extends through the disks 5 at, for example, the central portions thereof. The disks 5 may be attached to the suture 4 by, for example a knot or metal piece, or may be strung on the suture 4 like, for example a button. The disks 5 will preferably be provided at fixed intervals on the suture 4 while loaded in the space 6, but all of the disks except for the first (5a) will be able to slide along the suture 4. The first disk 5a will be fixed in position on the suture 4. In addition, while loaded in the space 6, the disks may be wrapped around the needle shaft 1a, as shown in FIG. 1.

The needle 1 should have a curved tip. Therefore, the outer casing 2 must be sufficiently flexible to be able to slide along the curved needle 1. It may be desirable for the tip to be steerable.

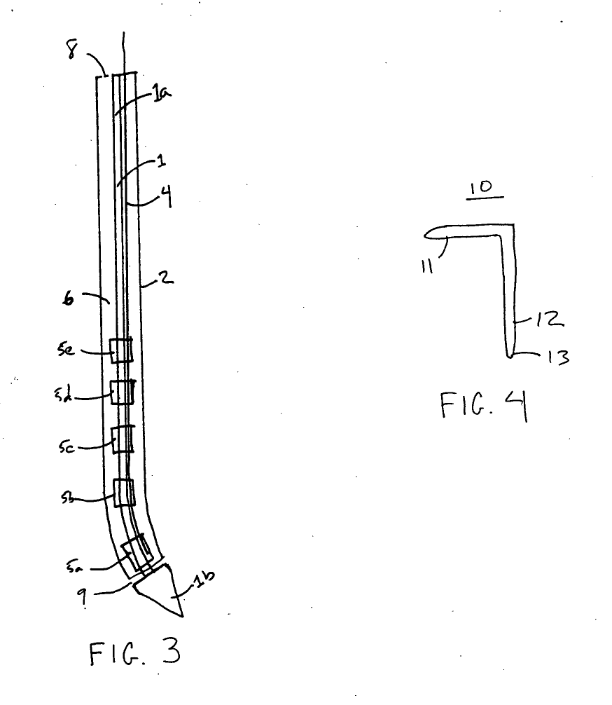

The suture 4 will preferably be Teflon, wire, or another permanent thread that is not degradable by acid. The disks 5 should be around 1 cm in diameter and should be pliable, such that the disks 5 can be wrapped around the needle 1 while held in the space between the needle 1 and the outer casing 2. The disks 5 may for example be polyurethane. And as shown in FIG. 2 the disks may include a thin metal support 7 in the shape of, for example, a rod or a rectangular thin metal piece.

The outer casing 2 may capable of screwing onto the needle 1 at the distal connection portion 3 by threads at the distal end of the outer casing 2 and at the distal connection portion 3 of the needle 1.

Alternatively, the outer casing 2 may be attached to the needle 1 at a proximal connection portion 8 (indicated by dashed lines in FIG. 3). With this structure, a narrow gap 9 would be provided between the outer casing 2 and the tip section 1b of the needle 1 to allow the suture 4 to pass therethrough. The outer casing 2 may be capable of screwing onto the needle 1 at the proximal connection portion 8.

Alternatively, as shown in FIG. 4, the outer casing 2 or the needle 1 may include an L-shaped groove 10 at the connection portion 8 and the other of the outer casing 2 and the needle 1 may include a projection for engaging with the groove. When the projection is engaged in the short part 11 of the L-shaped groove 10, the outer casing 2 will not be able to retract with respect to the tip section 1b of the needle. When the projection is engaged in the long part 12 of the L-shaped groove 10, the outer casing 2 will be retractable. The long part 12 includes a stop 13 at the end thereof to stop the projection when the projection is engaged in the long part 12, to prevent the outer casing 2 from being retracted too far and allowing more than one disk 5 to be discharged.

Alternatively, as shown in FIG. 5, the distal connection part 3 of the needle 1 may include a groove 14 in the tip section 1b of the needle. The outer casing 2 would be able to enter the groove 14 to seal the space 6.

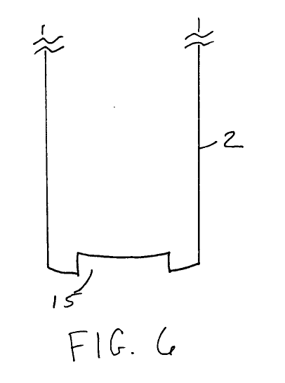

Preferably, with any structure in which the outer casing 2 connects to the needle 1 at the distal connection part 3, the outer casing will include at least one window 15 (see FIG. 6) to allow the suture 4 to pass therethrough. The window 15 should not be large enough to allow one of the disks 5 to escape from the space 6.

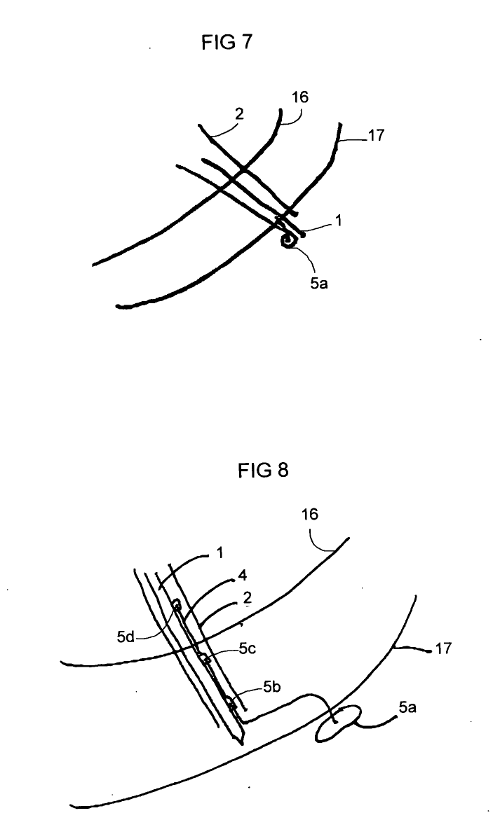

The stomach lumen restriction procedure may be performed under CT guidance while the patient is on the CT table. As shown in FIG. 7, the device will be inserted percutaneously and through both the near and far walls 16 and 17 of the stomach, while the stomach is inflated to a necessary degree. Once the tip of the device extends past the far wall 17 of the stomach, the outer casing 2 will be detached from the distal connection portion 3 of the needle 1 and retracted to allow the disk 5a to fall out from the space 6 between the outer casing 2 and the needle shaft 1a. The outer casing 2 will be retracted only enough to discharge a single disk.

It may be possible to use the metal support 7 in the disk 5a wrapped around the needle 1 as a spring to provide force to discharge the disk 5a from its position wrapped around the needle 1 when the outer casing is retracted so as to no longer surround the disk 5a. With this structure, the disks 5a-5e would not be completely wrapped around the needle 1, but rather the outer portions of the disks 5a-5e would press against the inner wall of the outer casing 2 while loaded in the space 6.

The outer casing 2 will then reattached back onto the needle 1 to prevent any further disks from falling out of the space 6. The tip of the device will then be withdrawn back into the stomach, leaving the disk 5a on the outside of the far wall 17 of the stomach, as shown in FIG. 8.

Then, the device will pierce through the stomach wall from the inside of the stomach at a second position at a distance from the first position. Once the tip of the device extends past the far wall 17 of the stomach, the outer casing 2 will be detached from the distal connection portion 3 of the needle 1 and retracted to allow the disk 5b to fall out from the space between the outer casing 2 and the needle 1. The outer casing 2 will then be reattached to the distal connection portion 3, and the tip of the device will then be retracted into the stomach, leaving the disk 5b on the outside of the stomach. Disks 5c and 5d will then be deposited outside of the stomach in a similar manner to disk 5b, such that nearly 360° of the stomach lumen is captured.

After depositing disk 5d, the device will be withdrawn completely from the stomach, and the outer casing 2 is unscrewed and retracted to allow the final disk 5e to fall out from the space between the outer casing 2 and the needle 1.

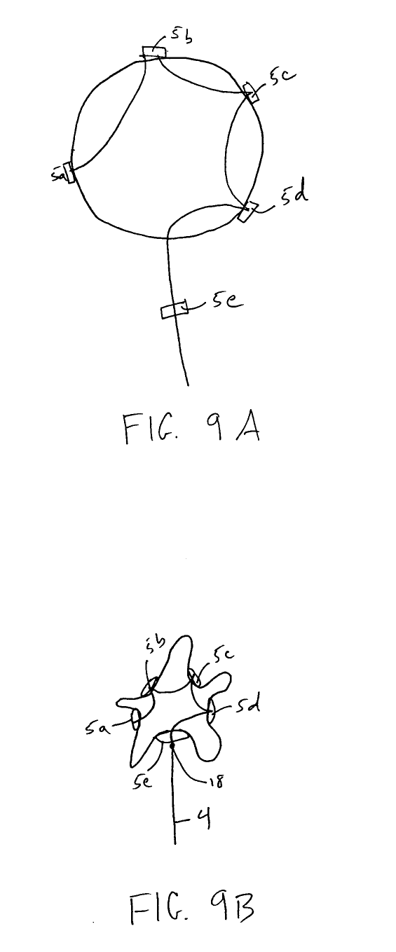

The final disk 5e will be slid down the suture 4 (see FIG. 9A) and the suture 2 will be pulled to draw the disks 5a-5e together along the suture 2 to narrow the stomach lumen to a desired size. That is, the suture 2 is cinched like a purse-string to draw the disks 5a-5e together along the suture to restrict the stomach lumen. The narrowed stomach lumen will still permit some food to pass to the distal stomach. As shown in FIG. 9B, a knot 18 will be made in the suture 4 and slid down the suture 4 to secure the final disk 5e against the outside of the stomach.

Although five disks 5a-5e are described above, it may be desirable to provide more or less than five disks. In addition, although only a curved needle tip is described above, the entire device may be curved to provide directionality, such that the device can be steered toward sites to be punctured. It may also be desirable for the needle tip to be steerable. Still further, the disks 5 may also be adjustably inflatable balloons, and tubing may be provided alongside the suture between the disks 5 to allow the disks 5 to be inflated and deflated. With this structure, an inflation port that is connected to the inflation tubing may be implanted subcutaneously such that the disks 5 will be inflatable and deflatable by injecting saline to the disks 5 via the inflation port and tubing. Yet still further, although the device is described above in connection with narrowing a stomach lumen, the device according to the first embodiment of the present invention could be used to provide a purse string type suture in other areas of the body.

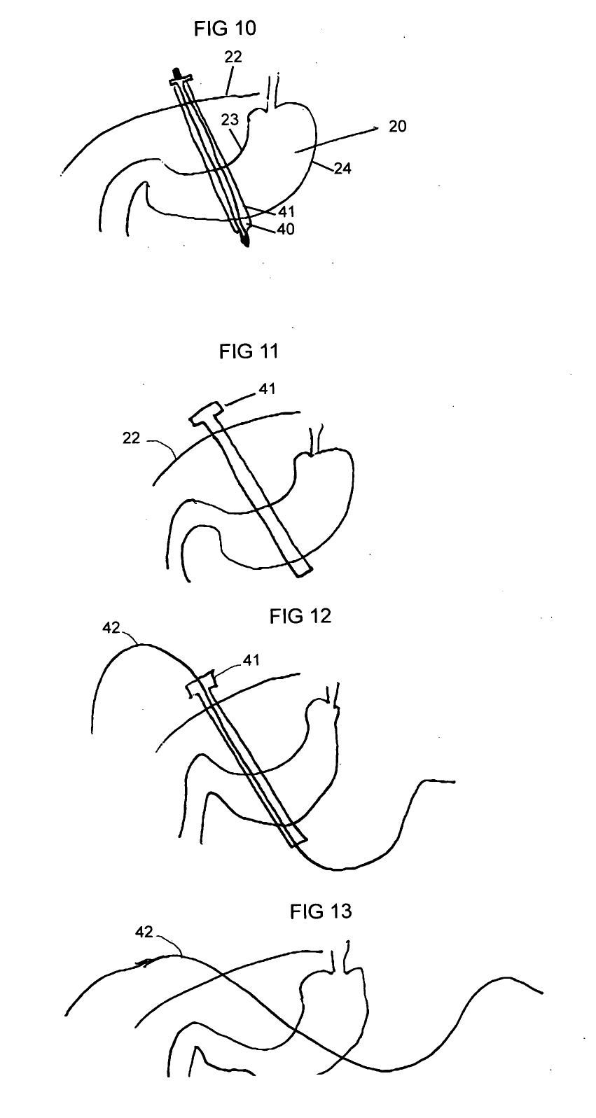

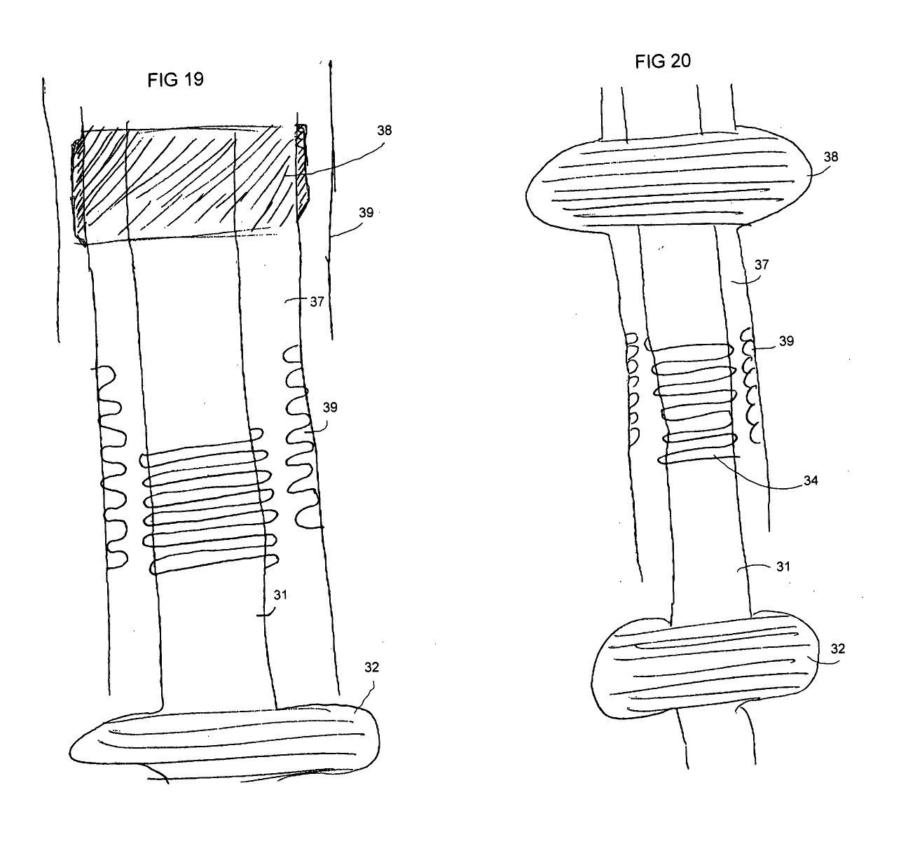

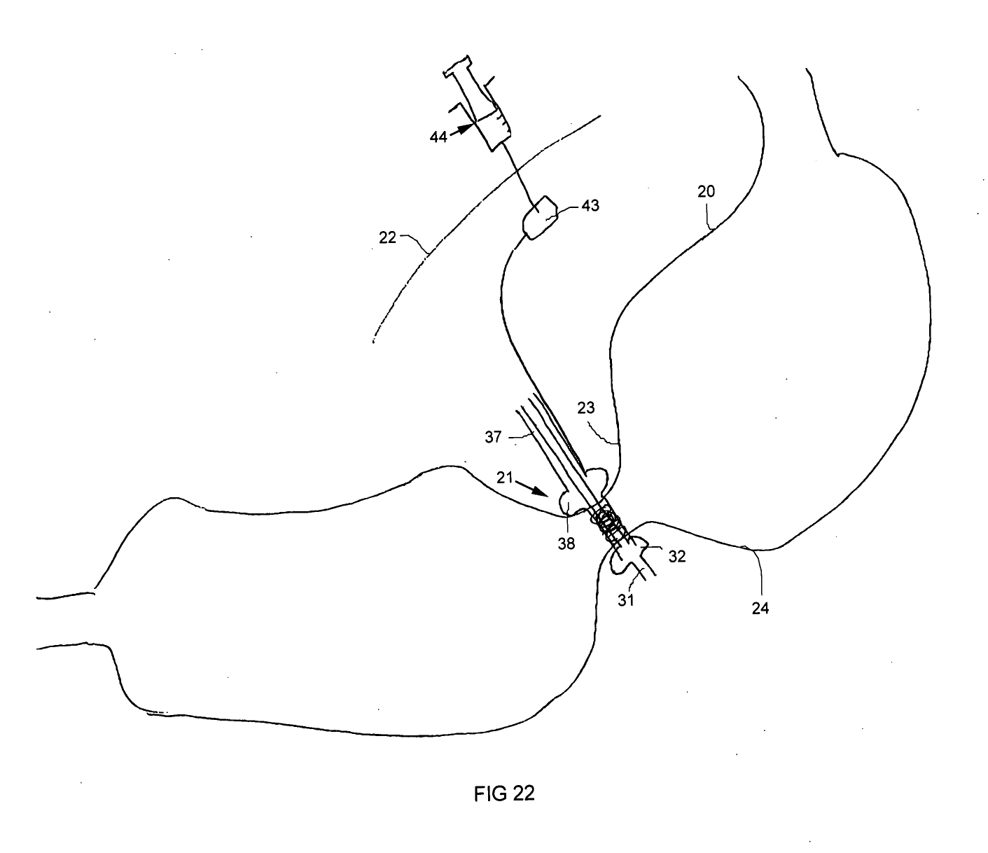

As shown in FIG. 22, a device for percutaneously restricting the stomach lumen according to a second embodiment of the present invention will include two balloons 32 and 38 which restrict the lumen of the stomach 20 at region 21. The insertion of the device according to the second embodiment is described in detail hereinbelow with respect to FIGS. 10-22.

The stomach lumen restriction according to the second embodiment may also be performed under CT guidance, with the stomach inflated to an appropriate degree to create a desired needle insertion “window.”

First, as shown in FIG. 10, a needle 40 with a trocar 41 will be inserted through the skin 22 and both the near wall 23 and the far wall 24 of the stomach 20. The needle 40 will be removed, leaving the trocar 41 in place, as shown in FIG. 11. Then, as shown in FIGS. 12 and 13, a wire 42 will be inserted through the trocar 41, and the trocar 41 will be removed, leaving the wire 42 in place.

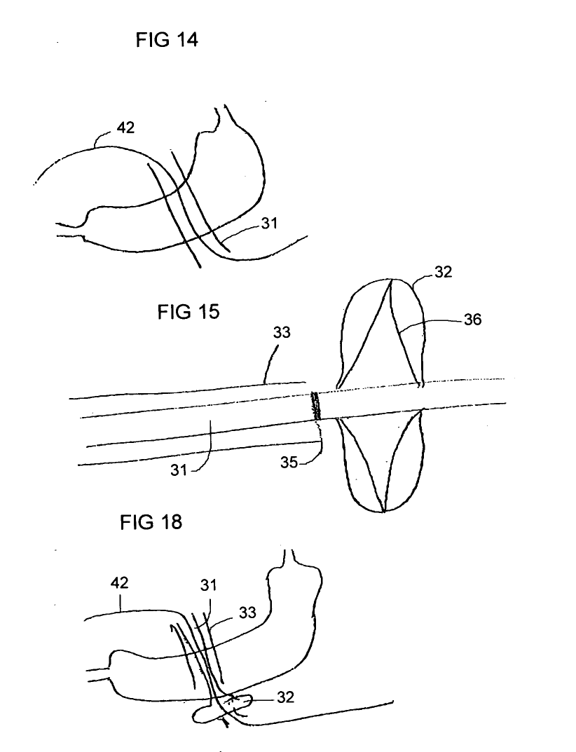

As shown in FIG. 14, a first catheter 31 will be guided along the wire 42 and inserted through both the near and far walls 23 and 24 of the stomach. As shown in FIG. 15, the first catheter 31 will include an outer casing 33 and the balloon 32. While the first catheter 31 is inserted, the outer casing 33 should cover the first catheter 31 substantially to the tip thereof to cover the balloon 32. When the end of the first catheter 31 including the balloon 32 has passed through the far wall of the stomach, the outer casing 33 is retracted from the tip of the first catheter 31, and the balloon 32. The metal marker 35 is provided for use in determining when the balloon 32 has completely passed through the far wall of the stomach.

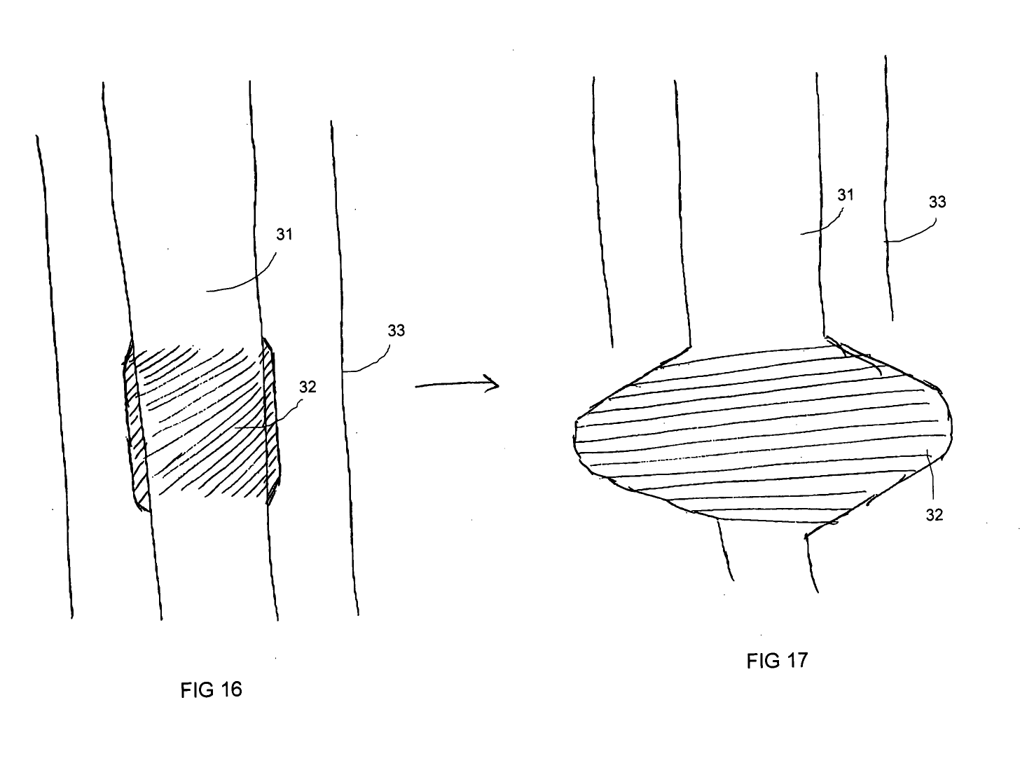

The balloon 32 may be expanded by a nitinol stent-like device 36 or by a saline infusion. FIGS. 16 and 17 are enlarged views of the outer casing 33 extending over the balloon 32 when not inflated (FIG. 16) and the balloon 32 in the inflated state after the outer casing 33 is retracted from the tip of the catheter 31 (FIG. 17). FIG. 18 shows the catheter 31 in position with the balloon 32 inflated.

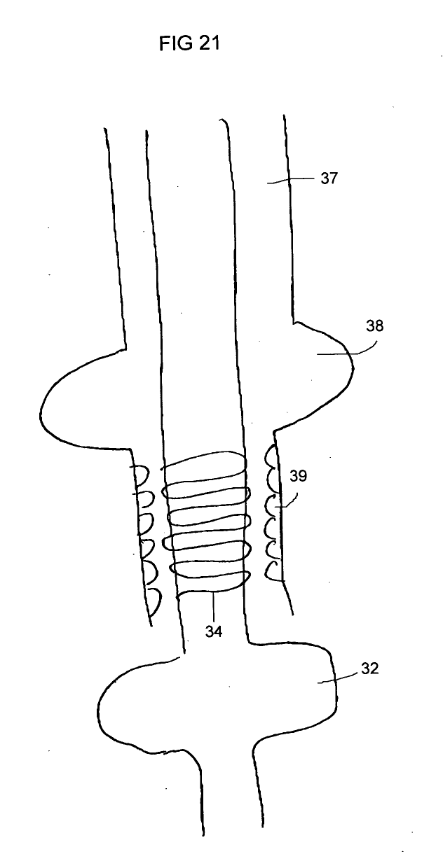

After the first catheter 31 is positioned, a second catheter 37, with balloon 38 and outer casing 39 will be guided over the wire and onto the first catheter 31. A distal end of the second catheter 37 will be inserted through at least the near wall 23 of the stomach. The second catheter 37 will be screwed onto the first catheter 31 using threads 34 and 39 on the first and second catheters 31 and 37. As shown in FIG. 20, once the threads 34 and 39 are engaged, the outer casing 39 of the second catheter 37 will be retracted, and the balloon 38 of the second catheter 37 will be inflated. As shown in FIG. 22, the balloon 38 of the second catheter 37 is provided outside the near wall 23 of the stomach. The second catheter 37 may be screwed down tightly onto the first catheter 31, as shown in FIG. 21, thereby cinching the near and far walls 23 and 24 of the stomach together.

The proximal ends of the first and second catheters 31 and 37 may be detachable from the distal ends thereof (including the balloons 32 and 38) by unscrewing the proximal ends from the distal ends. The proximal ends of the catheter will then be removed from the patient, leaving the distal ends of the first and second catheters 31 and 37 in place as shown in FIG. 18.

An inflation port 43 may be implanted beneath the skin 22 of the patient, to allow a corresponding one of the balloons 32 and 38 to be inflated and deflated by injecting saline into the inflation port using a syringe 44. Alternatively, both of the balloons 32 and 38 may be inflated and deflated via the inflation port 43, or two inflation ports may be provided to allow both balloons 32 and 38 to be individually adjusted. The inflation port 43 is connected to the balloon(s) 32 and 38 via tubing 45. The width of the stomach at the narrowed region 21 would thereby be adjustable.

The foregoing provides a detailed description of presently preferred embodiments. Various modifications and additions can be made without departing from the spirit and scope of the invention. Accordingly, the foregoing description is meant to be taken only by way of example and not to otherwise limit the scope of the present invention as defined in the appended claims.

1. A device for percutaneously restricting a lumen, comprising:
a needle;
an outer casing which surrounds the needle to define a space between the outer casing and the needle;
a suture provided in the space between the outer casing and the needle; and
a plurality of disks provided along the suture;
wherein the needle is adapted to pierce a wall of the lumen at a plurality of positions;
wherein the outer casing is retractable with respect to the needle to provide an opening through which the disks individually exit the space between the outer casing and the needle at each of the plurality of positions; and
wherein the lumen is restricted by drawing together the disks along the suture.

2. A device for percutaneously restricting a lumen, comprising:
a first catheter comprising a first inflatable balloon portion at a distal end thereof;
a second catheter comprising a second inflatable balloon portion at a distal end thereof;
wherein the second catheter is adapted to be coupled to the first catheter such that when the second catheter is coupled to the first catheter the first balloon portion and the second balloon portion are provided at an adjustable distance with respect to each other along a lengthwise direction of the catheters.

3. A method for percutaneously restricting a lumen, comprising:
piercing a wall of the lumen from within the lumen at a plurality of positions;
depositing a disk outside of the wall of the lumen at each of the plurality of positions, the disks being connected along a suture; and
restricting the lumen by drawing together the disks along the suture.
